(19) United States
(12) Patent Application Publication
(10) Pub. No.: US 2024/0247237 A1
(43) Pub. Date: Jul. 25, 2024

(54) SCALABLE PRODUCTION METHOD FOR AAV

(71) Applicant: The Trustees of the University of Pennsylvania, Philadelphia, PA (US)

(72) Inventors: Martin Lock, Southampton, PA (US); Luc H. Vandenberghe, Weston, MA (US); James M. Wilson, Philadelphia, PA (US)

(21) Appl. No.: 18/475,942

(22) Filed: Sep. 27, 2023

Related U.S. Application Data
(63) Continuation of application No. 17/806,005, filed on Jun. 8, 2022, now abandoned.
Application No. 17/806,005 is a continuation of application No. 16/998,548, filed on Aug. 20, 2020, now Pat. No. 11,377,641.
Application No. 16/998,548 is a continuation of application No. 16/179,322, filed on Nov. 2, 2018, now abandoned.
Application No. 16/179,322 is a continuation of application No. 14/919,801, filed on Oct. 22, 2015, now Pat. No. 10,155,931.
Application No. 14/919,801 is a continuation of application No. 12/226,588, filed on Oct. 21, 2008, now Pat. No. 9,198,984.
(60) Provisional application No. 60/796,229, filed on Apr. 28, 2006.

(51) Int. Cl. C12N 7/00; A61K 48/00; C07K 14/005

(52) U.S. Cl. CPC C12N 7/00; A61K 48/0091; C07K 14/005; C12N 2750/14151

(57) ABSTRACT

A method for producing AAV, without requiring cell lysis, is described. The method involves harvesting AAV from the supernatant. For AAV having capsids with a heparin binding site, the method involves modifying the AAV capsids and/or the culture conditions to ablate the binding between the AAV heparin binding site and the cells, thereby allowing the AAV to pass into the supernatant, i.e., media. Thus, the method of the invention provides supernatant containing high yields of AAV which have a higher degree of purity from cell membranes and intracellular materials, as compared to AAV produced using methods using a cell lysis step.

### CROSS-REFERENCE TO RELATED APPLICATIONS

This is a continuation of U.S. patent application Ser. No. 17/806,005, filed Jun. 8, 2022, which is a continuation of U.S. patent application Ser. No. 16/998,548, filed Aug. 20, 2020, which is a continuation of U.S. patent application Ser. No. 16/179,322, filed Nov. 2, 2018, which is a continuation of U.S. patent application Ser. No. 14/919,801, filed Oct. 22, 2015, now U.S. Pat. No. 10,155,931, issued Dec. 18, 2018, which is a continuation of U.S. patent application Ser. No. 12/226,588, filed Oct. 21, 2008, now U.S. Pat. No. 9,198,984, issued Dec. 1, 2015, which is a national stage application under 35 U.S.C. 371 of PCT/US2007/010055, filed on Apr. 27, 2007, now expired, and which claims the benefit of U.S. Patent Application No. 60/796,229, filed Apr. 28, 2006, now expired.

### REFERENCE TO AN ELECTRONIC SEQUENCE LISTING

The contents of the electronic sequence listing (UPN-S4197USC5.xml, size: 33,273 bytes; and Date of Creation: Apr. 17, 2024) is herein incorporated by reference in its entirety.

### BACKGROUND OF THE INVENTION

This invention describes a novel way to harvest and produce AAV.

Adeno-associated virus (AAV), a member of the Parvovirus family, is a small nonenveloped, icosahedral virus with a single-stranded linear DNA genome of 4.7 kilobases (kb) to 6 kb. AAV is assigned to the genus, Dependovirus, because the virus was discovered as a contaminant in purified adenovirus stocks. AAV's life cycle includes a latent phase at which AAV genomes, after infection, are integrated into host genomes and an infectious phase in which, following either adenovirus or herpes simplex virus infection, the integrated AAV genomes are subsequently rescued, replicated, and packaged into infectious viruses. The properties of non-pathogenicity, broad host range of infectivity, including non-dividing cells, and integration make AAV an attractive delivery vehicle.

A variety of different AAV sequences and methods for isolating same from tissues have been described. AAV1-6, AAV7, AAV9 and AAV9, amongst other AAV sequences obtained from simian or human tissue sources have been described. See, e.g., International Patent Publication Nos. WO 02/33269, WO 02/386122 (AAV8), and International Patent Publication No. WO 2005/033321. With this, a move away from defining AAV strictly by serologic cross-reactivity (serotypes) has occurred. Recent literature defines the relationship between these AAV in terms of phylogenetic relatedness, proposing groups termed “clades”. See, e.g., Gao et al, J Virol, 78(12):6381-6388 (June 2004); International Patent Publication No. WO 2005/033321.

Current methodology for production of AAV has been founded largely in view of the observation that AAV2 is cell-associated and thus, thought to reside primarily in the producing cells. Therefore most current state-of-the-art AAV production strategies obtain vector particles from the cellular pellet of the production cell line. Each of these strategies employs some methodology of releasing vector from the cell pellet by sonication, enzymatic, chemical or physical lysis. This unfortunately releases all intracellular proteins and debris into the viral harvest. Therefore the subsequent purification procedure is more demanding. Because of the relatively low efficiency of both production and purification, it is necessary to start with a large amount of producing cells.

What are needed are efficient methods of production and purification of AAV.

### SUMMARY OF THE INVENTION

The present invention provides a method for producing AAV, without requiring the termination of the virus-producing cell culture. The method involves harvesting AAV released into the supernatant without requiring collection of a cell pellet or cell disruption. In one embodiment, the method involves modifying the AAV capsids, the cells, and/or the culture conditions to substantially reduce or eliminate binding between the AAV heparin binding site and the producer cells, thereby allowing the AAV to pass into the supernatant, i.e., media. Thus, the method of the invention provides supernatant containing high yields of AAV which have a higher degree of purity from cell membranes and intracellular materials, as compared to AAV produced using methods using a cell collection and/or cell lysis step.

This technology can be applied for efficient and scalable AAV production with significant improvements in financial and time cost. This technology allows small scale AAV production and its commercial application for an all-inclusive kit for research purposes. Since AAV can be harvested multiple times from the supernatant, a continuous system or bioreactor allows production of particle amounts necessary for clinical use or wide pharmaceutical application without cellular substrate being the limitation for production. Optionally in combination with the use of a growth medium that is absent or low in serum or proteins, purification is significantly simplified. This technology can be applied in combination with more efficient methods of purification and concentration than could be used with the prior art production methods and/or in the presence of significant amounts of intracellular material.

Still other advantages of the present invention will be apparent from the detailed description of the invention.

### BRIEF DESCRIPTION OF THE DRAWINGS

FIG. 1 illustrates the effect of a heparan sulfate glycoprotein (HSPG) on production of AAV2 and AAV2HSPG- in the presence (S) or absence of serum (SF) with AAV2/8 production as positive control.

FIG. 2A shows quantification of DNase-resistant AAV particles (drp) in supernatant (I), loosely cell associated (II) and tightly cell associated (III) fraction for AAV2/8 positive control.

FIGS. 2B-2E show similar fractions for AAV2 and AAV2HSPG- in the presence (Serum or S) or absence (Serum Free or SF) of serum.

FIG. 3 illustrates the yield of AAV isolates from a single 15 cm dish transfection. All non-heparin binding isolates yield higher than 2×10¹² GC ranging up to 1×10¹³ GC total. AAV2 and isolate hu.51 has been shown to bind heparin and is limited in production.

FIG. 4 is a bar chart showing the results of immunization with a variety of AAV on T-cell activation. Balb/c mice were immunized with 1×10¹¹ GC AAV2/6, AAV2/6.1, AAV2/6.2, AAV2/6.1.2, AAV2/1 and AAV2 vector. 13 days later splenocytes were harvested from 3 mice per group and pooled. Equal amounts of splenocytes were stimulated in vitro with the Balb/c AAV epitope IPQYGYLTL [SEQ ID NO: 1] in a ELISPOT assay.

FIG. 5 is a bar chart showing the fraction of vector released into supernatant following production of the specified AAV viral vector by triple transient transfection in a 293 cell culture system.

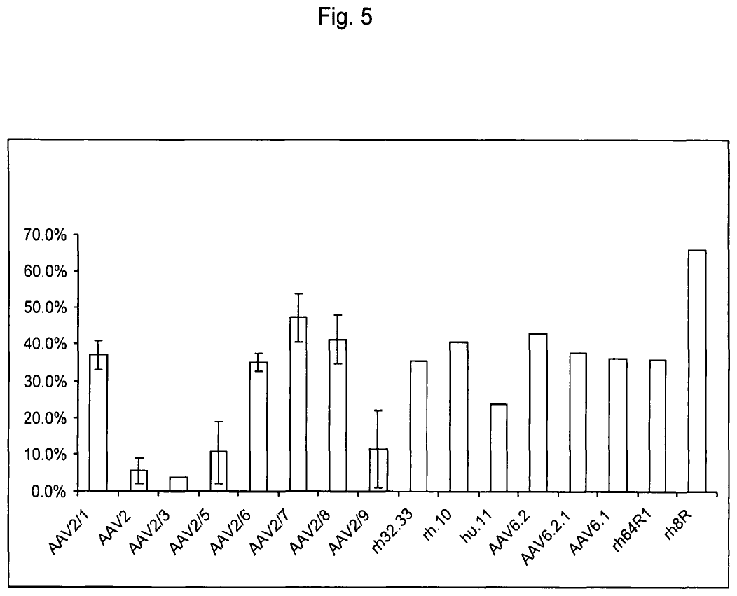

### DETAILED DESCRIPTION OF THE INVENTION

The present invention provides a method for producing AAV, without requiring cell disruption. The method involves harvesting AAV from the supernatant of a viral production culture.

For those AAVs that do not show affinity with heparan-sulfate proteoglycan or heparin, which comprises the majority of AAV species, a large fraction of DNase resistant, infectious particles is located in the culture supernatant or is only loosely cell-associated. This is observed without induced viral, osmolytic or any other type of lysis.

This invention allows a scalable technology for production of AAV for use in a variety of gene transfer and/or vaccine applications. It also dramatically reduces the stringency of purification when used in combination with low- or non-protein contaminating media for harvest. This production method can be applied in combination with suitable methods for purification and concentration including, e.g., chromatography, filtration or precipitation for purification and concentration.

Since most current AAV production strategies use the cell pellet as a substrate for isolating particles, such methods are by definition an iterative process that excludes a continuous harvesting strategy.

In contrast to current methodologies, in one embodiment, the present invention provides a method in which the supernatant is the major source for many AAVs. This allows repetitive or continuous harvesting of the same producer cells for production of larger amounts of AAV for clinical or pre-clinical research or therapy. In the current cell pellet harvest and subsequent purification methods, large amounts of particles are needed to efficiently provide a usable viral titer. Therefore there is a threshold below which recovery of a usable amount of particles is technically not feasible. In one embodiment, an AAV vector secretes at least about 10% Dnase-resistant vector particles or genomes (drp vg) from the cells in which it is produced. Such drp vg represent genomic sequences (e.g., a minigene, cassette, and/or AAV nucleic acid sequences) packaged in AAV capsid. In other embodiments, an AAV vector secretes at least about 20% drp vg. In still other embodiments, an AAV vector secretes at least about 40% drp vg. By the more efficient production strategy provided herein, scalability is possible for both small and large particle needs. Therefore viral production can be customized depending on expected quantities required, without the requirement for cell lysis or discontinuing the cell culture.

For example, AAV8 vectors have been found to secrete, on average, more than 40% of their viral particles into the supernatant in a 293 cell-based triple transfection production method. Other vectors based on AAV7 and rh8R have been found to secrete in this same range. Still other vectors have been found to secrete, on average, more than 30% of the viral particles in the supernatant in this system, e.g., AAV1 [capsid protein provided in SEQ ID NO: 2], AAV6 [AAV6 capsid protein provided in SEQ ID NO: 3], AAV6.1 [SEQ ID NO: 3, with a K531E change in the capsid protein], AAV6.1.2 [SEQ ID NO: 3, with K531E,F129L], rh.32.33 [capsid protein provided in SEQ ID NO: 4], rh.10 [capsid protein provided in SEQ ID NO: 5], and rh64R1 [rh64 capsid protein provided in SEQ ID NO: 6, with a R697W] and rh8R [rh8 capsid protein provided in SEQ IDNO: 7, with D531E]. In yet another example, other AAV vectors have been found to secrete on average more than 20% of their viral particles into the supernatant during production following triple transfection in this system. Still other AAV vectors, e.g., those based on AAV9 [capsid protein provided in SEQ ID NO: 8], have been found to release more than 10% of their viral particles into the supernatant in this system. Yet other examples include AAVs which secrete more than 10% of their viral particles into the supernatant are used in the methods of the invention. In one embodiment, these vectors produced in this matter are from AAVs which naturally secrete out of the cell in which they are produced.

In another embodiment, the AAVs are modified to permit their secretion. In one embodiment, the inventors have found that an AAV having a heparin binding domain and which is characterized by having transduction (infectious) ability blocked by heparin, do not secrete in detectable amounts. Examples of such AAV are AAV2 [capsid protein provided in SEQ ID NO: 9], which is mostly cell associated during production, and AAV3 [capsid protein provided in SEQ ID NO: 10]. Thus, in one embodiment, the method involves modifying the AAV capsids, the cells, and/or the culture conditions to substantially reduce or eliminate binding between the AAV heparin binding site and the producer cells, thereby allowing the AAV to pass into the supernatant, i.e., media.

The method of the invention provides supernatant containing high yields of AAV which have a higher degree of purity from cell membranes, proteins, and intracellular materials, as compared to AAV recovered following cell lysis. In one embodiment, this invention in contrast starts from supernatant without lysis and thereby simplifies any subsequent purification. Limited amount of cell debris is found in the supernatant in normal culture and amounts to a dramatic reduction of protein contamination. In one embodiment, serum-free medium is utilized to avoid the contaminating effect of the serum or other proteins introduced by the growth medium.

In one aspect, the present invention provides a method of producing an AAV in a viral production culture. The sequences of a variety of AAV have been previously described. See, e.g., AAV 1 (U.S. Pat. No. 6,759,237), AAV2, AAV3, AAV4, AAV5, AAV6, AAV7, AAV8, AAV9, rh32.33, rh.10, hu.11, others AAV from human and non-human sources, see, e.g., International Patent Publication Nos. WO 02/33269, WO 02/386122 (AAV8), and GenBank, and such sequences as have been altered to correct singleton errors, e.g., AAV6.2, AAV6.1, AAV6.1.2, rh64R1 and rh8R [see, e.g., WO 2006/110689, published Oct. 19, 2006]. Alternatively, other AAV sequences including those identified by one of skill in the art using known techniques [See, e.g., International Patent Publication No. WO 2005/033321 and GenBank] or by other means may be modified as described herein.

Certain AAV sequences are natively devoid of such a heparin binding site. For AAV lacking a heparin binding site, e.g., AAV8 [capsid protein provided in SEQ ID NO: 11], no modification of the AAV sequence, cell or media is required. The ability of an AAV capsid to bind heparin can be readily identified using a variety of assay formats and heparin or portions thereof for binding an AAV. Further, the ability of heparin to block the infectious/transduction ability of an AAV can readily be determined by one of skill in the art. A suitable assay for determining the ability of heparin to block any infection/transduction of transduction of an AAV has been described, e.g., in C. Halbert et al, J Virol, 75(14):6615-6624 (July 2001) and C. E. Walsh and H. Chao, Haemophilia, 8 (Suppl. 2), p. 60-67 (2002).

Other AAV sequences, e.g., AAV6, have a heparin binding site, but the ability of AAV6 to infect is partially inhibited, not blocked, by the presence of heparin. The AAV6 vp1 capsid sequence has been described as having a single amino acid residue that mediates heparin binding, the native lysine reside at position 531 [SEQ ID NO: 3]. [The sequence of AAV6 is provided in International Patent Appln No. PCT/US06/13375 and the residue number is based on the numbering scheme provided in that international application (see, e.g., Table)]. In such a case, no modification of this AAV sequence is required since it has been found to be only loosely cell associated.

In one embodiment, for AAV having a heparin binding site and which has its ability to infect/transfect cells blocked by heparin, the invention provides for modification of the AAV to reduce or eliminate heparin-binding in order to increase the amount of viral particles secreted into the supernatant. In one embodiment, a heparin binding domain is an Arg-Xaa-Xaa-Arg (RxxR) [SEQ ID NO: 12] motif as has been described in AAV2 (i.e., about amino acids 585 to 588 of the AAV2 vp1 capsid protein, SEQ ID NO: 9, Kern, et al., J Virol 77:11072-81; Opie, et al., J Virol 77:6995-7006 (based upon the numbering illustrated in WO 02/33269)]. Xaa represents any amino acid. The inventors have identified other AAV capsids having RxxR motifs, several of which are Clade B AAVs. Examples of such AAV capsids having RxxR motifs include, hu.51 [SEQ ID NO: 13], hu.34 [SEQ ID NO: 14], hu.35 [SEQ ID NO: 15], hu.45 [SEQ ID NO: 16], and hu.47 [SEQ ID NO: 17]. Other AAV having an RxxR domain can be readily identified by one of skill in the art from among those AAV sequences which have been described. In addition, other heparin binding sites can be readily identified in AAV using techniques known to those of skill in the art. In another example, AAV3 binds heparin; however, it does not contain the RxxR domain.

The inventors have found that by changing an amino acid residue(s) of a heparin binding sequence to contain a non-conservative amino acid change, not only is heparin binding ablated, but also, T cell activation is significantly reduced. This is the subject of the co-owned application, “Modified AAV Vectors Having Reduced Capsid Immunogenicity and Use Thereof”, which claims priority of U.S. provisional Patent Application No. 60/795,965, filed Apr. 28, 2006, which is hereby incorporated by reference.

In one embodiment, the invention provides a method for producing an AAV in a viral culture, wherein the AAV is modified to ablate the heparin binding domain.

In one embodiment, the nucleic acid sequence encoding the AAV capsid heparin binding site is modified using site-specific mutagenesis techniques, in which the codon for the amino acid residue(s) responsible for mediating heparin binding is altered to make a non-conservative change in the encoded amino acid. Examples of non-conservative amino acid changes include those, e.g., substitution of one amino acid with another amino acid of different chemical structure (properties), which affect protein function. The following table illustrates the most common amino acids and their properties.

| | | | | | Aromatic | |
|---|---|---|---|---|---|---|
| Amino | | Hydro- | Po- | | or | |
| acid | Abbrev. | phobic | lar | Charged | Aliphatic | Codon |
| Alanine | Ala, A | X | — | — | — | GCU, GCC, |
| | | | | | | GCA, GCG |
| Cysteine | Cys, C | X | — | — | — | UGU, UGC |
| Aspartate | Asp, D | — | X | negative | — | GAU, GAC |
| Glutamate | Glu, E | — | X | negative | — | GAA, GAG |
| Phenyl- | Phe, F | X | — | — | Aromatic | UUU, UUC |
| alanine | | | | | | |
| Glycine | Gly, G | X | — | — | — | GGU, GGC, |
| | | | | | | GGA, GGG |
| Histidine | His, H | — | X | positive | Aromatic | CAU, CAC |
| Isoleucine | Ile, I | X | — | — | Aliphatic | AUU, AUC, |
| | | | | | | AUA |
| Lysine | Lys, K | — | X | positive | — | AAA, AAG |
| Leucine | Leu, L | X | — | — | Aliphatic | UUA, UUG, |
| | | | | | | CUU, CUC, |
| | | | | | | CUA, CUG |
| Methionine | Met, M | X | — | — | — | AUG |
| Asparagine | Asn, N | — | X | — | — | AAU, AAC |
| Proline | Pro, P | X | — | — | — | CCU, CCC, |
| | | | | | | CCA, CCG |
| Glutamine | Gln, Q | — | X | — | — | CAA, CAG |
| Arginine | Arg, R | — | X | positive | — | CGU, CGC, |
| | | | | | | CGA, CGG, |
| | | | | | | AGA, AGG |
| Serine | Ser, S | — | X | — | — | UCU, UCC, |
| | | | | | | UCA, UCG, |
| | | | | | | AGU, AGC |
| Threonine | Thr, T | X | X | — | — | ACU, ACC, |
| | | | | | | ACA, ACG |
| Valine | Val, V | X | — | — | Aliphatic | GUU, GUC, |
| | | | | | | GUA, GUG |
| Tryptophan | Trp, W | X | — | — | Aromatic | UGG |
| Tyrosine | Tyr, Y | X | X | — | Aromatic | UAU, UAC |

For example, the nucleic acid sequence encoding the heparin binding site is modified using site-specific mutagenesis techniques. For example in an RxxR motif [SEQ ID NO: 3], the codon for the initial arginine and/or the last arginine of the motif is altered to change one (or both) of the amino acids to a non-conservative amino acid. It has been found that altering either one of the arginines in this motif prevents heparin binding. As illustrated herein, where the heparin binding motif is RxxR, the first amino acid of the modified heparin sulfate glycoprotein binding site can be changed from Arg to Ser or Glu. In another embodiment, the last amino acid of the modified heparin sulfate glycoprotein binding site is changed from Arg to Thr. In another embodiment, the lysine at position 531 of the AAV6 vp1 capsid protein [SEQ ID NO: 3] is changed to a non-conservative amino acid. Non-conservative amino acid changes other than those illustrated herein may be selected by one of skill in the art.

Similarly, other heparin binding domains may be identified using techniques known to those of skill in the art and modified using site-specific mutagenesis or another suitable technique for altering the coding sequence for the arginine. See, e.g., Sambrook et al, Molecular Cloning: A Laboratory Manual, Cold Spring Harbor Press (Cold Spring Harbor, NY).

Additionally, other methods of altering the sequence of a heparin binding domain may be utilized to prevent heparin binding. In another embodiment, binding of heparin to an AAV containing a heparin binding site is ablated by methods other than altering the sequence of the heparin binding site. For example, one may provide the AAV capsid with a molecule which effectively masks the heparin binding site in the producer cell.

In yet another embodiment, one may modify the producer cell to eliminate or substantially reduce heparin production, e.g., using RNA targeted to or mutating genes important for heparin biogenesis, either transiently or permanently. In another embodiment, a producer cell line naturally defective in heparin biogenesis might be used.

A viral cell culture utilizes cells containing, either stably or transiently, at least the minimum components required to generate an AAV particle, where production of an AAV DNase resistant genome containing particles involves packaging an expression cassette into an AAV capsid. The minimum required components include, an expression cassette to be packaged into the AAV capsid, an AAV cap, and an AAV rep or a functional fragment thereof, and helper functions.

A variety of suitable cells and cell lines have been described for use in production of AAV. The cell itself may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, W138, HeLa, a HEK 293 cell (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc.

AAV sequences may be obtained from a variety of sources. For example, a suitable AAV sequence may be obtained as described in WO 2005/033321 or from known sources, e.g., the American Type Culture Collection, or a variety of academic vector core facilities. Alternatively, suitable sequences are synthetically generated using known techniques with reference to published sequences. Examples of suitable AAV sequences are provided herein.

Generally, the expression cassette is composed of, at a minimum, a 5′ AAV inverted terminal repeat (ITR), a nucleic acid sequence encoding a desirable therapeutic, immunogen, or antigen operably linked to regulatory sequences which direct expression thereof, and a 3′ AAV ITR. In one embodiment, the 5′ and/or 3′ ITRs of AAV serotype 2 are used. However, 5′ and 3′ ITRs from other suitable sources may be selected. It is this expression cassette that is packaged into a capsid protein to form an AAV virion (particle).

In addition to the expression cassette, the cell contains the sequences which drive expression of an AAV capsid in the cell (cap sequences) and rep sequences of the same source as the source of the AAV ITRs found in the expression cassette, or a cross-complementing source. The AAV cap and rep sequences may be independently selected from different AAV parental sequences and be introduced into the host cell in a suitable manner known to one in the art. While the full-length rep gene may be utilized, it has been found that smaller fragments thereof, i.e., the rep78/68 and the rep52/40 are sufficient to permit replication and packaging of the AAV.

The cell also requires helper functions in order to package the AAV of the invention. Optionally, these helper functions may be supplied by a herpesvirus. In another embodiment, the necessary helper functions are each provided from a human or non-human primate adenovirus source, such as are available from a variety of sources, including the American Type Culture Collection (ATCC), Manassas, VA (US). The sequences of a variety of suitable adenoviruses have been described. See, e.g., chimpanzee adenovirus C1 and C68 [U.S. Pat. No. 6,083,716]; Pan 5, Pan6 and Pan7, [WO 02/33645], hybrid adenoviruses such as those described [e.g., WO 05/001103], and GenBank.

In one embodiment, the host cell contains at least the minimum adenovirus DNA sequences necessary to express an Ela gene product, an E1b gene product, an E2a gene product, and/or an E4 ORF6 gene product. The host cell may contain other adenoviral genes such as VAI RNA, but these genes are not required. In one embodiment, the cell used does not carry any adenovirus gene other than E1, E2a and/or E4 ORF6; does not contain any other virus gene which could result in homologous recombination of a contaminating virus during the production of rAAV; and it is capable of infection or transfection by DNA and expresses the transfected gene (s).

One cell useful in the present invention is a host cell stably transformed with the sequences encoding rep and cap, and which is transfected with the adenovirus E1, E2a, and E40RF6 DNA and a construct carrying the expression cassette as described above. Stable rep and/or cap expressing cell lines, such as B-50 (International Patent Application Publication No. WO 99/15685), or those described in U.S. Pat. No. 5,658,785, may also be similarly employed. Another desirable host cell contains the minimum adenoviral DNA which is sufficient to express E4 ORF6. Yet other cell lines can be constructed using the novel modified cap sequences of the invention.

The preparation of a host cell according to this invention involves techniques such as assembly of selected DNA sequences. This assembly may be accomplished utilizing conventional techniques. Such techniques include cDNA and genomic cloning, which are well known and are described in Sambrook et al., Molecular Cloning: A Laboratory Manual, Cold Spring Harbor Press, Cold Spring Harbor, NY., including polymerase chain reaction, synthetic methods, and any other suitable methods which provide the desired nucleotide sequence.

The required components for AAV production (e.g., adenovirus E1a, E1b, E2a, and/or E40RF6 gene products, rep or a fragment(s) thereof, cap, the expression cassette, as well as any other desired helper functions), may be delivered to the packaging host cell separately, or in combination, in the form of any genetic element which transfer the sequences carried thereon.

As used herein, a genetic element (vector) includes, e.g., naked DNA, a plasmid, phage, transposon, cosmid, episome, a protein in a non-viral delivery vehicle (e.g., a lipid-based carrier), virus, etc., which transfers the sequences carried thereon. The selected vector may be delivered by any suitable method, including transfection, electroporation, liposome delivery, membrane fusion techniques, high velocity DNA-coated pellets, viral infection and protoplast fusion. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY. See, e.g., K. Fisher et al, J. Virol., 70:520-532 (1993) and U.S. Pat. No. 5,478,745.

In one embodiment, one or more of the adenoviral genes are stably integrated into the genome of the host cell or stably expressed as episomes. The promoters for each of the adenoviral genes may be selected independently from a constitutive promoter, an inducible promoter or a native adenoviral promoter. The promoters, for example, may be regulated by a specific physiological state of the organism or cell (i.e., by the differentiation state or in replicating or quiescent cells) or by exogenously added factors.

In one embodiment, a stable host cell will contain the required component(s) under the control of a regulatable promoter. However, the required component(s) may be under the control of a constitutive promoter.

Regulatable promoters allow control of gene expression by exogenously supplied compounds, environmental factors such as temperature, or the presence of a specific physiological state, e.g., acute phase, a particular differentiation state of the cell, or in replicating cells only. Regulatable promoters and systems are available from a variety of commercial sources, including, without limitation, Invitrogen, Clontech and Ariad. Many other systems have been described and can be readily selected by one of skill in the art. Examples of promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Harvey et al, Curr. Opin. Chem. Biol., 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. Still other types of inducible promoters which may be useful in this context are those which are regulated by a specific physiological state, e.g., temperature, acute phase, a particular differentiation state of the cell, or in replicating cells only.

In another embodiment, the native promoter is used. The native promoter may be used when it is desired that expression of the gene product should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

Another embodiment of the transgene includes a transgene operably linked to a tissue-specific promoter. For instance, if expression in skeletal muscle is desired, a promoter active in muscle should be used. These include the promoters from genes encoding skeletal β-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., J Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J Bone Miner. Res., 11:654-64 (1996)), lymphocytes (CD2, Hansal et al., J Immunol., 161:1063-8 (1998); immunoglobulin heavy chain; T cell receptor a chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others.

Examples of suitable activatable and constitutive promoters are known to those of skill in the art. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

Alternatively, one or more of the components required to be cultured in the host cell to package an expression cassette in an AAV capsid may be provided to the host cell in trans using a suitable genetic element.

Once a suitable cell culture system is selected, the cells are cultured in a suitable media under conditions which permit packaging of the AAV, the supernatant is collected from the culture, and the AAV isolated therefrom. In one embodiment, the invention provides a system which is scalable, allowing a cell culture to be maintained throughout a continuous production process, i.e., not requiring cell disruption and/or cell lysis for collection. In one embodiment, such a system maintains a viable cell culture. In another embodiment, the cell culture contains a mixed population of viable and non-viable cells. During the culture process, media can be added during the culturing process and/or in conjunction with collection of the supernatant to provide a continuous production process. This addition of media, fresh cells, and/or required nutritional or other elements such as a regulating agent may be repeated at least two times, from two to 100 times, or more than 100 times, depending upon the life of the cell culture.

While the method of the invention permits continuous production of the virus, upon completion of the production run, it may be desirable to extract any AAV remaining from the production cells prior to destruction thereof. This extraction can be performed using methods commonly used therefor. Such methods typically include removing the supernatant, lysing the cells by freeze/thaw or sonication techniques, following by detergent treatment (e.g., benzonase). Purification is traditionally performed by three rounds of CsCl gradient centrifugation, dialysis and concentration.

In one embodiment, the invention provides a cell culture containing cells grown in suitable culture media. Optionally, any components necessary to activate or induce expression of a desired gene product or required for virion production are supplied prior to, or at appropriate times during, production. Such components may be added with the media or supplied separately. For example, one or more suitable genetic element (e.g., a plasmid) carrying a required component(s) may be transfected into the desired cell line.

In one embodiment, the medium is a serum-free media such as Dulbecco's Modified Eagle Medium (DMEM), which contains such inorganic salts as CaCl₂(anhyd.), Fe(NO₃) 9H₂O, KCl, MgSO₄(anhyd.), NaCl, and NaH₂PO₄H₂O, amino acids such as L-arginine HCl, L-cystine 2HCl, glutamine, glycine, histidine HCl H₂O, isoleucine, lysine HCl, methionine, phenylalanine, serine, threonine, tryptophan, tyrosine 2Na 2H₂O, and valine, vitamins such as D-Ca pantothenate, choline chloride, folic acid, i-inositol, niacinamide, riboflavin, and thiamine HCl, and other components such as D-glucose, phenol red, and sodium pyruvate. Other suitable serum-free media may be selected.

A cell produced in the absence of serum (i.e., in serum-free medium) according to the invention, preferably has the additional advantage that it can be cultured in the absence of serum or serum components. Thus, isolation is easy and cost effective, safety is enhanced, and the system has good reliability (synthetic media are the best for reproducibility). The cells of the invention and, in particular, those based on primary cells, are capable of normal (for humans) post- and peri-translational modifications and assembly. This means that they are very suitable for preparing viral proteins and viruses for use in therapeutic and vaccine compositions.

In another embodiment, media containing serum may be selected. Additionally or alternatively, media may be mixed before or during the culturing process with desired nutrients, activating (inducing) agents, or serum (e.g., DMEM+10% fetal bovine serum). In still another embodiment, protein free media may be utilized.

Fresh media and any necessary inducing or activating agents are supplied (e.g., by a peristaltic pump or other suitable means) and spent media is removed from the culture vessel at the same flow rate. The culture volume is maintained and the dilution rate can be altered by changing the pump running speed. After initiation, the culture is maintained at a suitable temperature range for the selected cell culture (e.g., about room temperature to 37° C.) with stirring. The culture may be aerobic or anaerobic, depending upon the selected cell type.

It is anticipated that media will need to be added approximately 24 hours following transfection, or initiation of culturing a stable expressing cell line. However, a culture can be periodically sampled to determine the concentrations of host cells and AAV in the supernatant, to assess more precisely the timing of supernatant collection and media addition.

Thus, in one embodiment, the invention provides a continuous system for AAV viral production. In one embodiment, a batch culture is used. For example, batch culture may utilize suspension and/or adherent cells, a fed-batch culture, fill and draw. A variety of batch culture systems are known to those of skill in the art and utilize, e.g., bioreactors, fermenters, micro-carrier systems, static flasks, cell factories, roller bottles, disposable bags (e.g., the Wave™ system), stainless steel and glass vessels. Other systems, e.g., perfusion systems such as hollow fibre bioreactors, Micro-carrier systems, cell cube system (Corning), spin filters, packed bed bioreactors (e.g., Fibre cell), an cell encapsulation, can be used for AAV viral production.

In a continuous system, it is well known to those of skill in the art to obtain samples at various stages and measure the concentration of the virus by infectivity, genome titration, or other suitable methods. Once the appropriate concentration is obtained, supernatant can be drawn into the desired purification system. At the same time, appropriate amounts of replacement media and any other necessary components are supplied to the cell culture.

The AAV in the supernatant can be harvested using suitable techniques which are known to those of skill in the art. For example, monolith columns (e.g., in ion exchange, affinity or IMAC mode), chromatography (e.g., capture chromatography, fixed method chromatography, and expanded bed chromatography), filtration and precipitation, can be used for purification and concentration. These methods may be used alone or in combination. In one embodiment, capture chromatography methods, including column-based or membrane-based systems, are utilized in combination with filtration and precipitation. Suitable precipitation methods, e.g., utilizing polyethylene glycol (PEG) 8000 and NH₃SO₄, can be readily selected by one of skill in the art. Thereafter, the precipitate can be treated with benzonase and purified using suitable techniques.

In one embodiment, advantageously, when produced using the method of the invention, the cell culture supernatant contains significantly higher levels of AAV as compared to AAV which remains within in the cells. In certain embodiments, the supernatant comprises AAV in a yield of at least 60%.

At present, the inventors have found that AAV production efficiency has been increased by harvest of supernatant versus cell pellet for more that 30 recombinant AAV species.

Thus, the invention also provides a virus for use in a therapeutic or vaccine composition obtainable by a method or by a use according to the invention, the virus or the viral protein being free of any non-human mammalian proteinaceous material and a pharmaceutical formulation comprising such a virus and/or viral protein. Examples of such viruses include those described in the co-owned patent application, entitled “Modified AAV Vectors Having Reduced Capsid Immunogenicity And Use Thereof”, filed on the same date herewith, and which claims the benefit of U.S. Provisional Patent Application No. 60/795,965, filed Apr. 28, 2006.

Thus, in one embodiment, the invention provides a kit for producing AAV as described herein. Such a kit may contain one or more of the following components. A suitable production cell capable of directing the packaging of an AAV viral particle may be supplied. Such a production cell may have been engineered to contain all of the elements required for production of the AAV. Alternatively, such a production cell may have been altered such that it lacks the ability to express heparin capable of binding to a heparin binding site. Other suitable components may include, a transfection reagent, a plasmid component for construction of a vector, a component necessary for collection, purification, concentration or harvesting of the assembled AAV particle, a reagent for negative or positive selection of viral particle in order to purify, a reagent for concentration of viral preparation, and/or a reagent for enzymatic digestion of contaminants in viral preparation.

The following examples are illustrative of methods for producing AAV particles in the supernatant of cell cultures according to the present invention.

### Example 1

### Experimentals:

293 cells were transfected with CaPO₄ with plasmids required for production of AAV, i.e., AAV2 rep, a adenoviral helper construct and a ITR flanked transgene cassette. The AAV2 rep plasmid also contains the cap sequence of the particular virus being studied. The cap sequence is the only variable in all experiments. These experiments have been repeated for several transgene cassettes. Twenty-four hours after transfection, which occurred in serum containing DMEM, the medium was replaced with fresh medium with or without serum. Three (3) days after transfection, a sample (I) was taken from the culture medium of the 293 adherent cells. Subsequently cells were scraped and transferred into a receptacle. After centrifugation to remove cellular pellet, a sample (II) was taken from the supernatant after scraping. Next cell lysis was achieved by three consecutive freeze-thaw cycles (−80° C. to 37° C.). Cellular debris was removed and sample (III) was taken from the medium. The samples were quantified for AAV by DNase resistant genome titration by Tagman™ PCR. The total production yield from such a transfection is equal to the particle concentration from sample III. Three fractions are contained in this namely the culture supernatant fraction, the cell pellet fraction and the fraction that is released by the scraping and subsequent centrifugation of the cells. The absolute numbers of these fractions are obtained in the following way.

$$Particle number supernatant = particle number sample I$$

$$Particle number fraction removed by scraping and spinning ( loosely cell associated ) = particle number sample II minus sample I$$

$$Particle number fraction in cell pellet = particle number sample III minus sample II .$$

### Results:

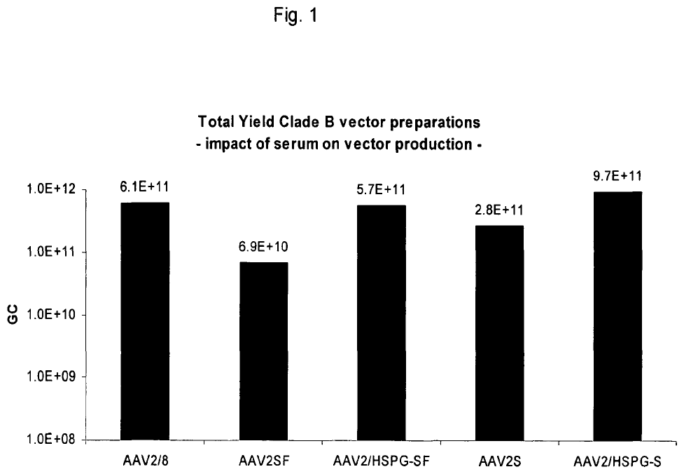

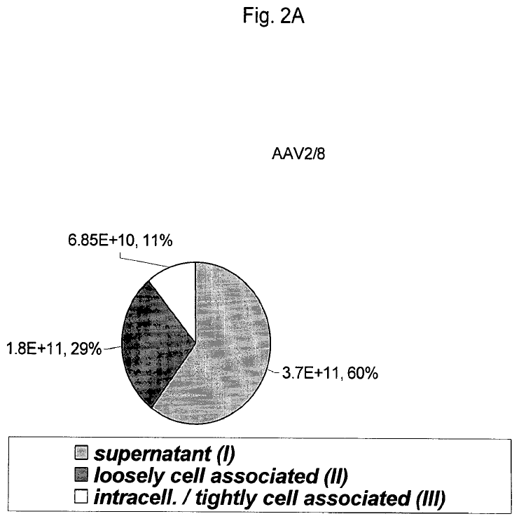

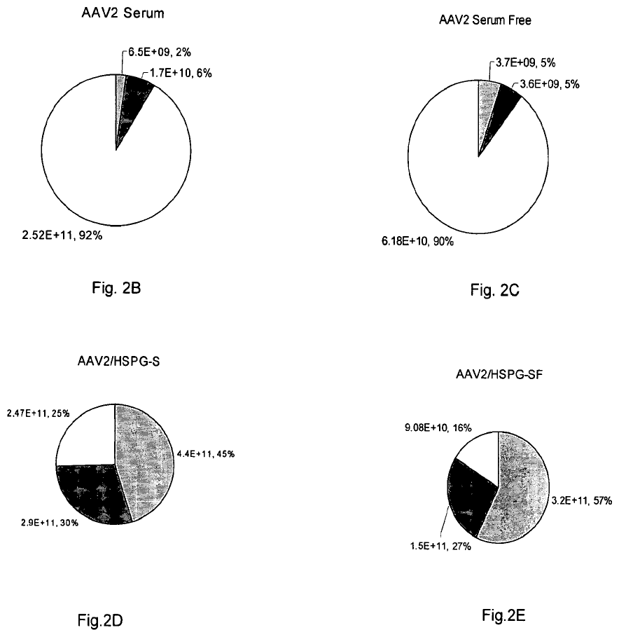

The presence of the RxxR [SEQ ID NO: 12] motif (domain) not only largely restricts the localization of the AAV particles to the cellular pellet but also limits its production from the cellular substrate possibly by saturation. This limitation is not observed for non-heparin binding AAV2 homologues or AAV8 (FIGS. 1 and 2). The presence (S) or absence (SF) of serum (FIG. 2) does not seem to dramatically impact on the production of AAV particles for the non-heparin binding AAV. The saturation effect of heparin binding AAV2 on the other hand seems to be alleviated some in the presence of serum.

In another example, using prior art methods at laboratory scale, about 40 15 cm dishes would be anticipated to yield on average for AAV2/7 around 4×10¹³ particles total. This current invention allows with inclusion of the supernatant to 4.7×10¹² particles to be harvested per plate.

Also, in combination with the use of serum-free medium, this technology reduces the subsequent purification effort dramatically. More particularly, AAV2/1 and AAV2/8 produced using the supernatant collection method of the invention were compared to AAV2/1 and AAV2/8 produced using previously described methods and purified via CsCl gradient. For both viruses, significantly higher infectivity was observed for the AAV2/1 and AAV2/8 particles obtained according to the supernatant collection method of the invention over a range of concentrations.

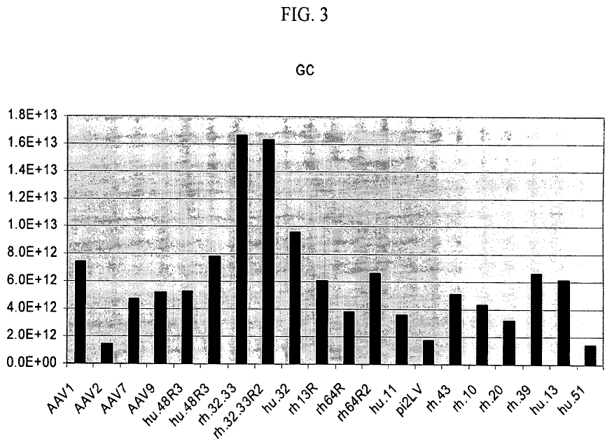

Reproducibly, for a large number of AAV isolates, DNase resistant particles from a single 15 cm dish transfection was produced with adeno-helper plasmid deltaF6, AAV rep-cap expressing trans plasmid for packaging and AAV2.CMV.eGFP cis plasmid for the vector genome. Titers for these small scale purifications for non-heparin binding isolates added up to 10¹² to 10¹³ genome copy containing particles per plate (FIG. 3). These quantities are sufficient for most lab applications for in vitro or in vivo experimentation.

### Example 2

Vector release into the supernatant was investigated for serotypes 1, 2, 3, 5, 6, 7, 8 and 9 as well as novel vectors rh32.33, rh.10, hu.11, AAV6.2, AAV6.1, AAV6.1.2, rh64R1 and rh8R. AAV2, AAV2/3 and AAV2/5 were found to secrete minimal (less than 10% of total Dnase-resistant vector genomes or particles (drp vg)). AAV2/9 is released moderately into the supernatant during viral vector production (more than 10%, less than 20% of total drp vg). All other vectors tested secrete more than 20% of their viral particles into the supernatant during production onto 293 cells following triple transfection.

The infectivity of vectors harvested from the supernatant was compared to that of purified (CsCl pure with the exception of AAV2 which was heparin purified) preparations as well as vector harvested from cellular pellet lysates. AAV harvested from the supernatant was found to be of equal or higher infectivity when compared to the two latter fractions in a 293 transduction assay for AAV vector based on isolate 1,2,6,8 or 9 as well as AAV2HSPG-.

Vector release into the supernatant seems to be correlated with its heparin affinity. Ablation of this affinity by genetically mutating the native AAV2 (SEQ ID NO: 9) RGNR [SEQ ID NO: 18] heparin binding motif (into SGNT, SEQ ID NO: 19) increases the fraction of vector that is released into the supernatant by more than 40%. The introduction of the heparin binding arginines at the homologous position on the AAV8 capsid (non-heparin binding, SEQ ID NO: 11) produces the AAV8RQNR vector that is almost entirely associated with the cellular pellet during the harvest of viral vector production. This is in contrast with its parental vector AAV8 which on average releases more than 40% of its retrievable vector genome particles into the supernatant.

### Example 3

An immunization study was performed to assess the effect of a variety of AAV vectors having differing capsids on T-cell activation. The study compared a native AAV6 capsid, known to have a heparin binding domain at the lysine residue at position 531 to three modified AAV having capsids with site-specific modifications introduced. These AAV, designated AAV2/6.2 (modified at a position other than K531), AAV2/6.1 (having an AAV6 capsid [SEQ ID NO: 3] modified at position 531 to contain a glutamic acid (i.e., a non-conservative amino acid change), and AAV2/6.1.2, having an AAV6 capsid with both the modifications of the AAV6.2 and AAV6.1 capsid were utilized. The sequences and generation of these vectors is described in International Patent Appln No. PCT/US06/13375. AAV1 served as a negative control and AAV2 served as a positive control.

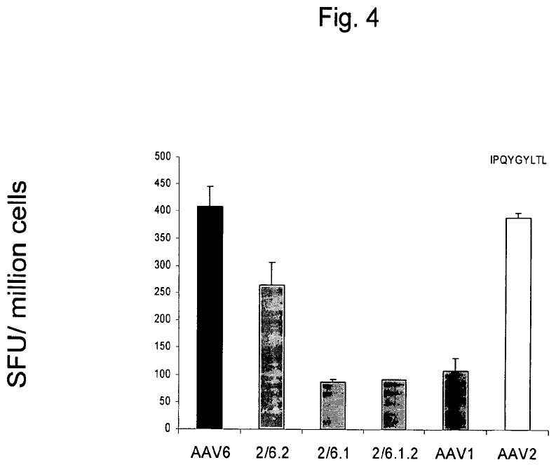

Balb/c mice (male) were immunized intramuscularly with 1×10¹¹ GC AAV2/6, AAV2/6.1, AAV2/6.2, AAV2/6.1.2, AAV2/1 or AAV2 vector. Thirteen (13) days later splenocytes were harvested from 3 mice per group and pooled. Equal amounts of splenocytes were stimulated in vitro with the Balb/c AAV epitope IPQYGYLTL [SEQ ID NO: 1] in a ELISPOT assay. See, FIG. 4.

These results show that viral vector containing an unmodified AAV6 capsid induced levels of T cells comparable to those induced by the AAV2 capsid. In contrast, the modified AAV6 vectors having ablated heparin binding domains (AAV2/6.1 and AAV2/6.1.2) had T-cell responses which are virtually indistinguishable from the negative control (AAV1).

This demonstrates that changing an amino acid residue responsible for mediating heparin binding to an AAV capsid to a non-conservative amino acid residue, not only ablates heparin binding, but also, significantly reduces T cell activation.

### Example 4

Several serotype AAVs were assessed for their ability to bind to an anion exchange membrane (Mustang Q, Pall Scientific) in buffers with pHs ranging from 6.0 to 9.0 and elution was monitored using a 0 to 500 mM salt gradient. High pH buffers were most suitable for the binding and elution of each of the serotypes tested (Table 1). Elution of three serotypes (AAV8, AAV7 and Rh8Rc) occurred in the 100 to 150 mM range of the gradient while two (AAV9 and Rh64R1) eluted directly following the application of the gradient. Recoveries of loaded material ranged from 50% (AAV7) to 100% (Rh64R1).

TABLE 1
Binding and Elution Characteristics of novel
AAV serotypes to Mustang Q Membranes

| | Optimal pH | | Optimal |
|---|---|---|---|
| | for Binding | Elution Start | Elution |
| | and Elution | Point (mM) | Yield |
| AAV 7 | 8 | 145 | 50% |
| AAV 8 | 9 | 110 | 71% |
| AAV 9 | 8.5 | 10 | 63% |
| Rh8Rc | 9 | 110 | 67% |
| Rh64R1 | 9 | 10 | 100% |

The data show that anion exchange membrane technology is applicable for the purification of a number of AAV serotypes. Due to the high flow rates and binding capacities afforded by the membranes macroporous structure, this technology is particularly suited for purification of AAV from cell culture supernatants. The data indicate that supernatant dilution or buffer exchange will be necessary to obtain appropriate salt concentrations for AAV binding to anion-exchange membranes.

All publications cited in this specification are incorporated herein by reference, as are the applications from which this application claims the benefit of priority: U.S. patent application Ser. No. 17/806,005, filed Jun. 8, 2022, U.S. patent application Ser. No. 16/998,548, filed Aug. 20, 2022, U.S. patent application Ser. No. 16/179,322, filed Nov. 2, 2018, U.S. patent application Ser. No. 14/919,801, filed Oct. 22, 2015; U.S. patent application Ser. No. 12/226,588, which has a 371 filing date of Oct. 21, 2008; PCT/US2007/01005, filed Apr. 27 2007, now expired; and U.S. Provisional Patent Application No. 60/796,229, filed Apr. 28, 2006, now expired, incorporated by reference herein. While the invention has been described with reference to particularly preferred embodiments, it will be appreciated that modifications can be made without departing from the spirit of the invention.

1. A method for production of AAV, said method comprising the step of:
(a) culturing an AAV lacking a heparin binding site in a cell culture;
(b) collecting supernatant from the culture; and
(c) isolating the AAV from the supernatant.


SEQUENCE LISTING (19 sequences)

SEQ ID NO: 1 - protein, 9 aa, unidentified
  REGION 1..9: note = Balb/c AAV epitope
IPQYGYLTL

SEQ ID NO: 2 - protein, 736 aa, Human adenovirus type 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - protein, 736 aa, Human adenovirus type 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 4 - protein, 733 aa, unidentified
  REGION 1..733: note = vp1, clone rh.32.33
MAADGYLPDWLEDNLSEGIREWWDLKPGAPKLKANQQKQDDGRGLVLPGYKYLGPFHGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEGEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRRNPEVQFTSNYGNQSSMLWAPDTTGKYTEPRVIGSRYLTNHL

SEQ ID NO: 5 - protein, 738 aa, unidentified
  REGION 1..738: note = vp1, clone rh.10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 6 - protein, 738 aa, unidentified
  REGION 1..738: note = vp1, clone rh.64
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRRNPEIQYTSNYYKSTNVDFAVNTEGVYSEPRPIGTRYLTRNL

SEQ ID NO: 7 - protein, 736 aa, unidentified
  REGION 1..736: note = vp1, clone rh.8
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 8 - protein, 736 aa, Human adenovirus type 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 735 aa, Human adenovirus type 2 [sequence omitted]

SEQ ID NO: 10 - protein, 736 aa, Human adenovirus type 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 11 - protein, 738 aa, Human adenovirus type 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 12 - skipped

SEQ ID NO: 13 - protein, 735 aa, unidentified
  REGION 1..735: note = vp1, clone hu.51
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVGEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSGYAHSQSLDRLMNPLIDQYLYYLSTTNTPSGTTTQSRLQFSQAGASDIRDQSRNWLPGPCYRQQRVSKTSADNNNSEYSWTGATKYHLNGRDSLVNPGPAMASHKDNEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 735 aa, unidentified
  REGION 1..735: note = vp1, clone hu.34
MAADGYLPDWLEDTLSEGIRQRWKLKPGPPPPEPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNESQAVGRSSFYCLEYFPSQMLRTGNNFTFSYTFEDVPFHSSYAHSQSLGRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 15 - protein, 735 aa, unidentified
  REGION 1..735: note = vp1, clone hu.35
MAADGYLPDWLEDTLSEGIRQRWKLKPGPPPPEPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLGRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - protein, 735 aa, unidentified
  REGION 1..735: note = vp1, clone hu.45
MAADGYLPDWLEDTLSEGIRQWWKLKPGPPPPKPAERHRDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSGYQLPYVLGSAHQGCLPPFPADVFMVPQYGYPTLNNGSQAVGRSSFYCLEYFPSQMLRTGNNFTFSYTFEDVPFHSSYAHSQSLDRLMNPLIDQYLYYLSTTNTPSGTTTQSRLQFSQAGASDIRDQSRNWLPGPCYRQQRVSKTSADNNNSEYSWTGATKYHLNGRDSLVNPGPAVASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 17 - protein, 735 aa, unidentified
  REGION 1..735: note = vp1, clone hu.47
MAADGYLPDWLEDTLSEGIRQWWKLKPGPPPPKPAERHRDDSRGLVLPGYKYLGPFNGLDKGEPVNEADAAALEHDKAYDRQLDSGDNPYLKYNHADAEFQERLKEDTSFGGNLGRAVFQAKKRVLEPLGLVGEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDSHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSTTNTPSGTTTQSRLQFSQAGASDIRDQSRNWLPGPCYRQQRVSKTSADNNNSEYSWTGATKYHLNGRDSLVNPGPAMASHKDNEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 18 - protein, 4 aa, unidentified
  REGION 1..4: note = chemically synthesized heparin binding domain
RGNR

SEQ ID NO: 19 - protein, 4 aa, unidentified
  REGION 1..4: note = chemically synthesized heparin binding domain
SGNT